(19) United States
(12) Patent Application Publication
(10) Pub. No.: US 2013/0253479 A1
(43) Pub. Date: Sep. 26, 2013

(54) MODIFIED FLOW CATHETERS AND METHODS OF USE THEREOF

(71) Applicant: Tsung-chow SU, Boca Raton, FL (US)

(72) Inventor: Tsung-chow SU, Boca Raton, FL (US)

(21) Appl. No.: 13/839,644

(22) Filed: Mar. 15, 2013

Related U.S. Application Data
(60) Provisional application No. 61/614,872, filed on Mar. 23, 2012.
(60) Provisional application No. 61/621,189, filed on Apr. 6, 2012.

(51) Int. Cl. A61M 25/00

(52) U.S. Cl. CPC A61M 25/0017
USPC 604/544

(57) ABSTRACT

A catheter includes an elongate body defining a continuous enclosed liquid flow channel, at least a portion of the liquid flow channel including a modification having a first surface and a second surface and an included volume between the first surface and the second surface, at least a portion of the included volume defining an elongated air flow channel, at least a portion of the first surface and the second surface coated with a hydrophobic (e.g., superhydrophobic) material. The catheter can be a urinary catheter (e.g., a urine drainage tube connected to a urine collection bag). Methods of flowing liquid through a catheter and of catheterizing a patient are encompassed.

### CROSS REFERENCE TO RELATED APPLICATIONS

This Application claims priority to U.S. Provisional Patent Application No. 61/614,872, filed Mar. 23, 2012, entitled “IMPROVED LIQUID FLOW THROUGH CATHETER TUBES WITH GEOMETRICAL AND SURFACE MODIFICATION”, and U.S. Provisional Patent Application No. 61/621,189, filed Apr. 6, 2012, entitled “IMPROVED LIQUID FLOW THROUGH CATHETER TUBES WITH GEOMETRICAL AND SURFACE MODIFICATION,” the entireties of which are incorporated herein by reference.

### FIELD OF THE INVENTION

This invention relates to apparatuses, devices and methods for improved liquid flow through catheters, and more particularly to modified urinary catheters and having improved liquid flow.

### BACKGROUND

Urine backflow is presently a major cause of catheter-associated urinary tract infections, which are the most common health-care associated infections world-wide. Approximately 15% to 25% of all patients that are admitted to hospitals will be catheterized at some point during their stay (Warren J. W., Journal of Anti-Microbial Agents, vol. 17, 299-303, 2001). Each subsequent day of catheter use is associated with an estimated 5% increase for risk of developing bacteriuria, and all catheterized patients will become bacteriuric if catheterized for long enough, even with excellent care. Infection due to catheterization is estimated to result in an additional 900,000 hospital days each year, and is the direct cause of 1,000 deaths, and 6,500 indirect deaths annually in the United States (Warren J. W., Journal of Anti-Microbial Agents, vol. 17, 299-303, 2001). These infections could be reduced by maintaining a closed drainage system, keeping high infection control standards, and preventing backflow from the catheter/bag.

However, in a closed drainage system, liquid cannot flow efficiently in a drainage tube since both ends of the tube are closed: one end is attached to the bladder and the other is connected to the drainage bag. Urine discharge is also slow and intermittent, and the drainage tube is occupied mostly by an air bubble. Urine must pass this air bubble in order to drain, and therefore urine must squeeze through the gap between the air bubble and the wall of the tube, as the air bubbles slowly rise and allow urine to pass. The resistance of this film flow is significant, and so the urine has a good chance to become stagnant and develop bacteria. This stagnant urine causes new urine flow to be forced to flow backwards, carrying the bacteria-ridden urine back to the urinary tract and causing infections.

U.S. Pat. No. 6,007,521 discloses a drainage catheter system which includes “a one-way fluid valve, a separation chamber, and a one-way gas valve” is U.S. Pat. No. 6,007,521. U.S. Pat. No. 5,531,724 uses a powder (already in the drainage bag) to change the urine from a liquid to a gel, thus reducing the possibility of backflow. A flutter valve is still necessary in this case. Due to the nature of the gel, the bags would necessarily be disposable and not reusable. U.S. Pat. No. 6,994,045 also uses super hydrophobic coatings to reduce drag. U.S. Pat. No. 3,604,420 uses geometrically-altered versions of the catheter tubes to eliminate negative pressure in the closed catheter drainage system.

At present, the most effective way to reduce the incidence of infection is to reduce the use of urinary catheters by restricting their use to patients who have clear indications and by removing the catheter as soon as it is no longer needed. Strategies to reduce the use of catheterization have been shown to be effective and are likely to have more impact on the incident of UTI's than any of the other strategies. As a general rule, the use of urinary catheters should be avoided if possible and be removed as soon as feasible due to the concern of infection.

### SUMMARY

A catheter as described herein includes an elongate body defining a continuous enclosed liquid flow channel, at least a portion of the liquid flow channel including a modification having a first surface and a second surface and an included volume between the first surface and the second surface, at least a portion of the included volume defining an elongated air flow channel, at least a portion of the first surface and the second surface coated with a hydrophobic (e.g., superhydrophobic) material. The modification and/or the hydrophobic material can have or impart anti-microbial (e.g., anti-bacterial) properties. In the catheter, the air flow channel excludes liquid flow but is in fluid communication with the liquid flow channel. The continuous enclosed liquid flow channel extends for the length of the elongate body, and the air flow channel is contiguous with the continuous liquid flow channel. The elongate body may define an inner wall and in one embodiment, the modification includes an elongated rod that is disposed within the continuous liquid flow channel and is attached to the inner wall, the rod being coated at least in part with the hydrophobic material. In other embodiments, the modification is a porous material, portions of the porous material defining the first and second surfaces and the air flow channel. The porous material can be a textile of interwoven fibers, the interwoven fibers defining the first and second surfaces and the air flow channel. The porous material can be, for example, a carbon foam. The porous material can define air passages having a size permitting the passage of air molecules and preventing the passage of water molecules. In an embodiment in which the elongate body defines an inner wall, the inner wall may have angular portions, the angular portions including the first and second surfaces. The elongate body may be triangular in cross-section, the corners of the triangle defining the angular portions. Alternatively, the elongate body may be circular in cross-section. The elongate body may include at least a first fin extending radially inward from the inner wall, portions of the at least first fin including at least one of the first and second surfaces. In this embodiment, at least a portion of the inner wall adjacent the at least one fin may include the hydrophobic coating, the included volume between the at least one fin and the inner wall defining the air flow channel. A catheter may further include at least a second fin adjacent and parallel to the at least first fin, the first fin defining one of the first and second surfaces, the at least second fin defining at least one of the first and second surfaces, the first fin and the second fin defining an enclosed space between the at least first and second fins, the enclosed space defining in part the air flow channel. The elongate body may include an inner wall including at least one elongated groove including the first and second surfaces, the groove and the first and second surfaces defining the air flow channel. In one embodiment, the catheter is a urinary catheter, and may further include an apparatus for retention of the catheter within a subject's urethra. In this embodiment, the catheter is dimensioned for insertion into a patient's urethra (e.g., having a diameter of about 0.15 to about 0.51 inches). The catheter may define proximal and distal ends, the retention apparatus being at the proximal end and a urine collection assembly in fluid communication with the distal end. The catheter can be a Foley catheter, for example. The catheter can be a drainage tube, such as one connected to a liquid collection bag (e.g., a urine drainage tube connected to a urine collection bag). In one embodiment, a urinary catheter as described herein includes an elongate body defining a continuous enclosed liquid flow channel, at least a portion of the liquid flow channel including a modification having a first surface and a second surface and an included volume between the first surface and the second surface, at least a portion of the included volume defining an elongated air flow channel, at least a portion of the first surface and the second surface coated with a hydrophobic material. A method of flowing liquid through a catheter includes providing a catheter as described herein, the catheter including opposing ends, an inlet orifice at one end and an outlet orifice at the other end; and causing liquid flow through the inlet orifice. In this catheter, air is trapped in the air flow channel and liquid flows through the liquid flow channel and not through the air flow channel. A method of catheterizing a patient includes providing a urinary catheter as described herein including opposing ends, an inlet orifice at one end and an outlet orifice at the other end; and positioning the inlet orifice in the patient's urethra, the outlet orifice in fluid communication with a urine collection device. In the methods, at least the one modification and/or the hydrophobic materials can impart anti-microbial (e.g., anti-bacterial) properties.

A modified catheter as described herein includes at least one modification that reduces impediment of liquid flow through the interior of the catheter (e.g., reducing or eliminating obstruction of liquid flow by air bubbles). In some embodiments, the modification is angular in geometry. In other embodiments, the modification may be a channel, perhaps C-shaped or U-shaped, or even a porous fabric. A feature common to all modifications of the modified catheters described herein is that two opposing hydrophobic or superhydrophic surfaces are spaced apart by a small enough distance that the repelling force of the surfaces does not allow liquid in between, creating a void space for the flow of air. These opposing hydrophobic or superhydrophobic surfaces continue for the length of the catheter such that air flow is not interrupted and air will flow the length of the catheter. Further, this air pocket/channel is in fluid communication with liquid (e.g., urine) flowing through the catheter.

Unless otherwise defined, all technical terms used herein have the same meaning as commonly understood by one of ordinary skill in the art to which this invention belongs.

As used herein, the term “superhydrophobicity” means a contact angle of greater than 140 degrees. The terms “hydrophobic” and “hydrophobic material” mean a material having a contact angle of 100-120 degrees. The term “intermediate superhydrophobicity” means a contact angle of at least 120-140 degrees. Accordingly, superhydrophobicity is a special case of hydrophobicity. In general, as contact angle increases, the void for air passage increases. This trend becomes very significant when the contact angle reaches 140 degrees or more.

By the term “catheter” is meant a hollow tube inserted into a body cavity, duct, or vessel to allow the passage of fluids or distend a passageway. Examples of catheters include urinary catheters (e.g., Foley catheters), drainage tubes and bags, drainage tubes and bags fluidly connected to a urinary catheter, etc.

The terms “patient,” “subject” and “individual” are used interchangeably herein, and mean a mammalian (e.g., human) subject to be treated and/or to obtain a biological sample from.

### BRIEF DESCRIPTION OF THE DRAWINGS

A fuller understanding of the present invention and the features and benefits thereof will be obtained upon review of the following detailed description together with the accompanying drawings, in which:

FIG. 1 is a plan view of a urinary Foley catheter.

FIG. 2 is a series of cross-sectional views of different embodiments of a catheter as described herein. FIG. 2A shows a triangular cross-section, FIG. 2B shows a circular cross-section, and FIG. 2C shows a fin wall cross-section.

FIG. 3 is a pair of cross-section views of additional embodiments of a catheter as described herein.

FIGS. 4-6 are formulas and graphs for the expelling of liquid surface from a high curvature location (e.g., a corner) in terms of contact angle.

FIG. 7 is a pair of schematic illustrations of liquid flow through a closed tube.

FIG. 8 is a pair of photographs of five cross-sectional areas used in the experiments described herein. The cross-sectional areas are: a circular cross section, a triangular cross-section, a fin walled cross-section, a helical threaded tube, and a circular tube with a rod placed on the inner surface. The ruler in the foreground provides a reference scale.

FIG. 9 is a graph showing the time taken for the fluid to travel one foot in a tube with both ends closed was measured in coated tubes of various cross section at various angles of inclination respective to the horizontal plane. No flow was observed in the tube with a circular cross-section.

FIG. 10 is a graph showing the time taken for the fluid to travel one foot in a tube with both ends open was measured in coated tubes of various cross section. Notice that the time taken drops significantly for tubes of the same geometry.

FIG. 11 is a graph showing the time taken for the fluid to travel in one foot in a tube with both ends open was measured for uncoated tubes of various cross section. In general, the time taken for the fluid to travel increased by a significant amount without hydrophobic coating.

FIG. 12 is a schematic illustration of cross-section views of a non-lined tube (left) and a tube having a superhydrophobic textile inserted within (right). In the non-lined tubes, no flow is possible at 90 degrees. When the superhydrophobic textile is inserted into the tubes, the water is able to flow.

FIG. 13 is a graph showing the effect of textiles on flow rate (speed) for rigid and flexible tubes.

FIG. 14 is a graph showing the effect of angle on flow rate (speed). This graph presents only the results for textile “A” for tubes “R9” and “F2” at all angles of inclination.

FIG. 15 is a schematic illustration of a metal wire placed into a catheter tube to pierce the fluid column and release the surface tension for dispersal of pressure.

FIG. 16 is a schematic illustration of a catheter with metal wire shown on the right and a catheter without the metal wire is shown on the left. It can be observed that the wire metal allows for dispersal of the fluid column, whereas the lack of wire metal in the catheter tube has caused the fluid to become obstructed in the form of a column.

FIG. 17 is a photograph of a drainage tube and bag. One end of the tube can be attached to a catheter.

FIG. 18 is a graph of the relation of max distance S and contact angle 0.

FIG. 19 is a set of formulas that describe the expelling of liquid surface from high curvature location (e.g., a corner) in terms of contact angle.

### DETAILED DESCRIPTION OF THE INVENTION

The invention provides devices, apparatuses and methods for improving liquid flow through a liquid flow device (e.g., a closed liquid flow system), such as a catheter, for use in any applications in which reduced frictional resistance between a liquid and the interior of the liquid flow device is desired. Examples of such applications include urinary catheters, dentistry drainage and/or irrigation devices, and closed system liquid flow devices used in low gravity or zero gravity conditions. The experimental results described herein relate to urinary catheters, urinary catheter drainage tubes, and catheter systems including urinary collection bags. Catheter-associated urinary tract infections could be reduced by maintaining a closed drainage system, keeping high infection control standards, and preventing backflow from the catheter/bag. However, in currently available closed drainage systems, liquid cannot flow efficiently in a drainage tube since both ends of the tube are closed: one end is attached to the bladder and the other is connected to the drainage bag. Urine discharge is also slow and intermittent, and the drainage tube is occupied mostly by an air bubble. Urine must pass this air bubble in order to drain, and therefore urine must squeeze through the gap between the air bubble and the wall of the tube, as the air bubbles slowly rise and allow urine to pass. The resistance of this film flow is significant, and so the urine has a good chance to become stagnant and develop bacteria. This stagnant urine causes new urine flow to be forced to flow backwards, carrying the bacteria-ridden urine back to the urinary tract and causing infections. The cross-sectional and surface properties of the modified catheters described herein prevent confined air bubbles from obstructing liquid flow by creating an air passage along the corner inside the tube.

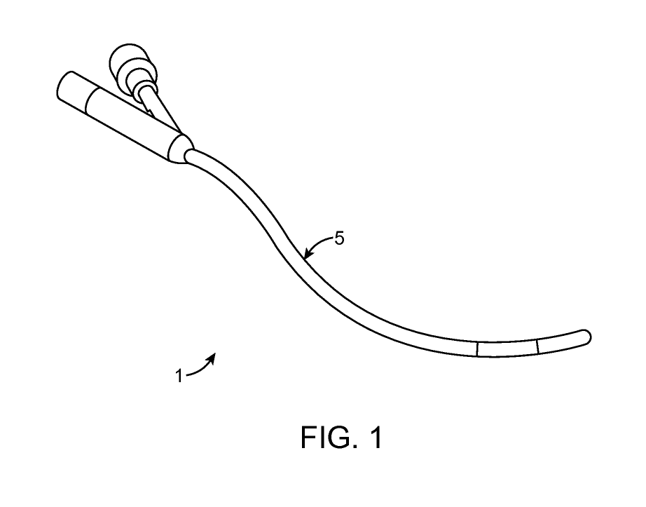

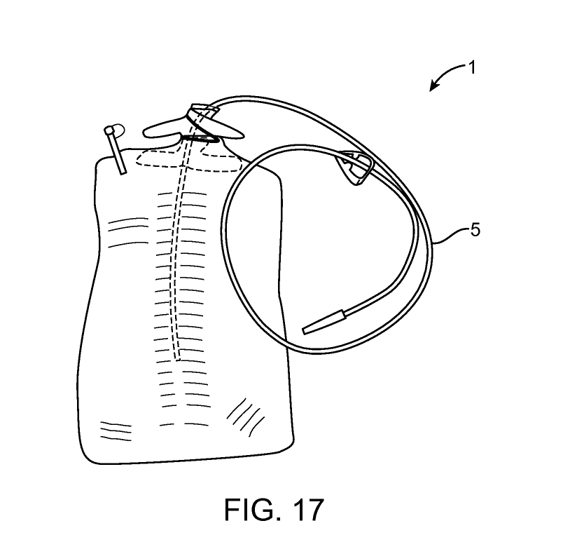

Referring to FIGS. 1 and 17, examples of a catheter (1) are shown. In FIG. 1, the catheter shown is a Foley catheter. In FIG. 17, the catheter shown is a drainage tube and bag (one end of the drainage tube able to be attached to a catheter). However, any type of catheter may be modified according to the methods and apparatuses described herein. A catheter as described herein is any hollow (typically thin) tube that allows drainage, and/or administration of fluids or gases. In most uses, a catheter is a thin, flexible tube (“soft” catheter), though catheters of varying levels of stiffness or rigidity are encompassed by the present invention. Catheters can be inserted into a body cavity, duct, or vessel. Some catheters can allow access by surgical instruments, and also perform a wide variety of other tasks depending on the type of catheter. The process of inserting a catheter is catheterization. In medicine, a catheter can be a thin tube extruded from medical grade materials serving a broad range of functions. In medicine, a catheter can also be catheter system that includes a catheter, a drainage tube, and a liquid collection bag (see, e.g., FIG. 17). Catheters include medical devices that can be inserted in the body to treat diseases (e.g., used to introduce fluids into a body) or perform a surgical procedure. By modifying the material or adjusting the way catheters are manufactured, catheters may be specifically designed for cardiovascular, urological, gastrointestinal, neurovascular, and ophthalmic applications. A catheter as described herein can be used in any application where a liquid flow through tube with at least one closed end (e.g., two closed ends) is desired.

In medicine, placement of a catheter into a particular part of the body allows for several applications. Examples of such applications include the following: draining urine from the urinary bladder as in urinary catheterization, e.g., the intermittent catheters or Foley catheter or even when the urethra is damaged as in suprapubic catheterization; drainage of urine from the kidney by percutaneous (through the skin) nephrostomy; drainage of fluid collections, e.g. an abdominal abscess; administration of intravenous fluids, medication or parenteral nutrition with a peripheral venous catheter; angioplasty, angiography, balloon septostomy, balloon sinuplasty, cardiac electrophysiology testing, and catheter ablation; direct measurement of blood pressure in an artery or vein; direct measurement of intracranial pressure; administration of anaesthetic medication into the epidural space, the subarachnoid space, or around a major nerve bundle such as the brachial plexus; administration of oxygen, volatile anesthetic agents, and other breathing gases into the lungs using a tracheal tube; and subcutaneous administration of insulin or other medications, with the use of an infusion set and insulin pump.

Typically, a catheter (1) includes an elongate body (5) defining a continuous enclosed liquid flow channel, at least a portion of the liquid flow channel including a modification having a first surface and a second surface and an included volume between the first surface and the second surface, at least a portion of the included volume defining an elongated air flow channel, at least a portion of the first surface and the second surface coated with a hydrophobic (e.g., superhydrophobic) material. In the catheter (1), the air flow channel excludes liquid flow but is in fluid communication with the liquid flow channel (20). The continuous enclosed liquid flow channel (20) extends for the length of the elongate body (5), and the air flow channel is contiguous with the continuous liquid flow channel (20). In a typical embodiment, the diameter of the elongate body (5) is less than about 0.365 inches and the length of the elongate body (5) is in the range of about 18 inches to about 84 (e.g., 17, 18, 20, 25, 30, 35, 40, 45, 50, 51, 55, 60, 68, 70, 75, 84, 85) inches.

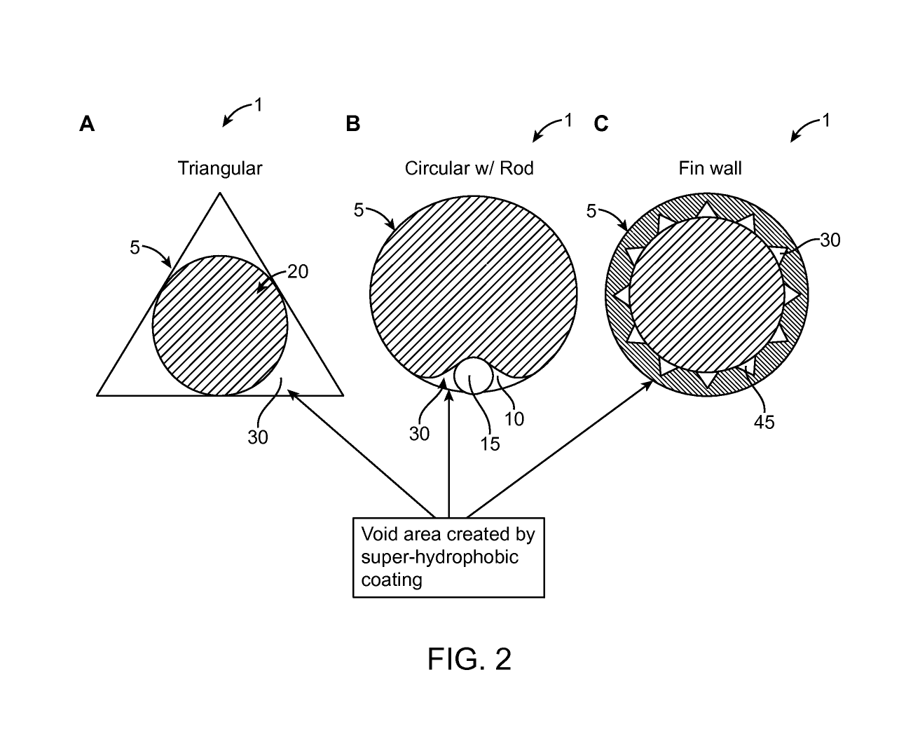

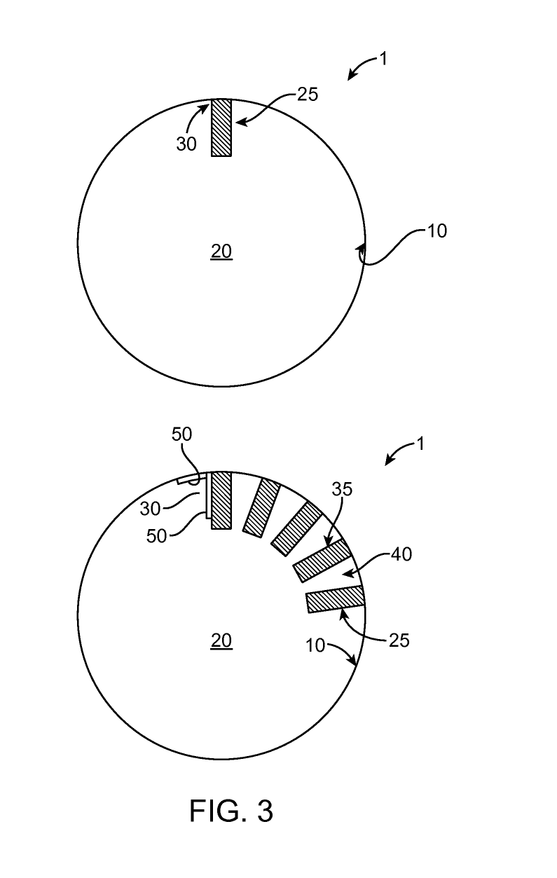

The elongate body (5) may define an inner wall and in one embodiment, as shown in FIG. 2, the modification includes an elongated rod (15) that is disposed within the continuous liquid flow channel (20) and is attached to the inner wall (10), the rod (15) being coated at least in part with the hydrophobic material. In other embodiments, the modification is a porous material, portions of the porous material defining the first and second surfaces and the air flow channel (30). The porous material can be a textile of interwoven fibers, the interwoven fibers defining the first and second surfaces and the air flow channel (30). The porous material can be any suitable porous material, for example, a carbon foam, and hydrophobic and superhydrophobic textiles such as Polypropylene spunband fabric or Polypropylene meltblown fabric. The porous material defines air passages having a size permitting the passage of air molecules and preventing the passage of water molecules. In an embodiment in which the elongate body (5) defines an inner wall (10), the inner wall (10) may have angular portions, the angular portions including the first and second surfaces. The elongate body (5) may be triangular in cross-section (FIG. 2A), the corners of the triangle defining the angular portions. Alternatively, the elongate body (5) may be circular in cross-section (FIG. 2B). The elongate body (5) may include at least a first fin (25) extending radially inward from the inner wall, portions of the at least first fin (25) including at least one of the first and second surfaces (FIG. 3). In this embodiment, at least a portion of the inner wall (10) adjacent the at least one fin (25) and at least a portion of the at least first fin (25) may include the hydrophobic coating (50), the included volume between the at least one fin (25) and the inner wall (10) defining the air flow channel (30). A catheter (1) may further include at least a second fin (35) adjacent and parallel to the at least first fin (25), the first fin (25) defining one of the first and second surfaces, the at least second fin (35) defining at least one of the first and second surfaces, the first fin (25) and the second fin (35) defining an enclosed space (40) between the at least first (25) and second fins (35), the enclosed space (40) defining in part the air flow channel. In another embodiment (FIG. 2C), the inner wall includes at least one elongated groove (45) including the first and second surfaces, the groove (45) and the first and second surfaces defining the air flow channel. A catheter as described herein may be sealed within packaging, such as packaging made from a liquid and gas impermeable material.

In one embodiment, the catheter is a urinary catheter, and may further include an apparatus for retention of the catheter within a subject's urethra. In this embodiment, the catheter is dimensioned for insertion into a patient's urethra. The dimensions of the catheter may depend upon the sex and size of the patient. Typically, the diameter of a catheter is about 0.13 to 0.365 inches. The catheter may define proximal and distal ends, the retention apparatus being at the proximal end and a urine collection assembly in fluid communication with the distal end. Any suitable retention apparatus can be used. For example, a balloon inflated with a sterile liquid may be used. In such an embodiment, the catheter can be a Foley catheter (see FIG. 1), for example. In an embodiment in which the catheter is a Foley catheter having an additional air flow channel for inflating the balloon that retains the catheter within the patient's urethra, the modification is present in the liquid flow channel of the catheter. Use and handling of urinary catheters, as well as urine collection devices and assemblies used in conjunction with urinary catheters, are well known in the art, and are described, for example, in U.S. Pat. Nos. 6,007,521 and 5,531,724, both incorporated herein by reference.

Any suitable hydrophobic (e.g., superhydrophobic) material can be used in the modified catheters described herein, particularly those having a contact angle greater than 90 degrees. For example, superhydrophobic textiles such as Polypropylene spunband fabric or Polypropylene meltblown fabric may be used. Additional examples of hydrophobic and superhydrophobic materials include Polystyrene, alkane-terminated self-assembled monolayers (SAMs), polydimethylsiloxane (PDMS), and Teflon (see, e.g., Dongqing Li (ed), Encyclopedia of Microfluidics and Nanofluidics, Springer; 2008 edition). Examples of superhdrophobic materials include perfluoroalkyl and perfluoropolyether superhydrophobic materials. Fabrication methods for producing superhydrophobic surfaces are known in the art, and include particle deposition, sol-gel techniques, plasma treatments,vapor deposition, and casting techniques. The inner walls and modifications of the catheters described herein can be coated with a proprietary superhydrophobic coating from Industrial Technology Research Institute(ITRI) Taiwan. This coating is a sol-gel coating containing trimethylsiloxane-silica nanoparticles. It has no known toxicity for external use. Its basic technology is similar to the coating composition described in US Patent Application Pub. No. US 2008/0221009A1. Another coating that can be used is a superhydrophobic coating composition with Sol-gel coating containing a mixture of hydroxyl terminated silica particles, a chemical modifying reagent, a linking agent, a cross-linking catalyst, and toluene or hexane (Zimmermann et al., Functional Materials, 2008, 18, 3662-3669). This coating is available through a company called Lotusleaf Coatings. In the approach of using air-breathable superhydrophobic textiles, a polyester (PET) material could be coated, resulting in a contact angle of 176 degrees (Mueller et al. Journal of Urology 2005; 173:490-2). The textile can be rolled into a tube and inserted into a catheter (e.g., a urine drainage tube). It may be possible that the air passage through the space within the air-breathable fabric would be sufficient and little or no additional modification may be required. Many superhydrophobic surfaces are a result of surface roughness, which entraps air, making surfaces superhydrophobic. Examples of additional hydrophobic materials are listed in Table 1 below.

TABLE 1
Examples of Critical Surface Tension and Contact Angle
with Water Measurements for Various Polymers

| | | | Contact |
|---|---|---|---|
| Polymer Name | CAS # | γs | Angle |
| Polyvinyl alcohol (PVOH) | 25213-24-5 | 37 | 51 |
| Polyvinyl acetate (PVA) | 9003-20-7 | 35.3 | 60.6 |
| Nylon 6 (polycaprolactum, aramid 6) | 25038-54-4 | 43.9 | 62.6 |
| Polyethylene oxide (PEO, PEG, | 25322-68-3 | 43 | 63 |
| polyethylene glycol) | | | |
| Nylon 6,6 | 32131-17-2 | 42.2 | 68.3 |
| Nylon 7,7 | — | 43 | 70 |
| Polysulfone (PSU) | 25135-51-7 | 42.1 | 70.5 |
| Polymethyl methacrylate (PMMA, acrylic, | 9011-14-7 | 37.5 | 70.9 |
| plexiglas) | | | |
| Nylon 12 | 24937-16-4 | 37.1 | 72.4 |
| Polyethylene terephthalate (PET) | 25038-59-9 | 39 | 72.5 |
| Epoxies | — | 44.5 | 76.3 |
| Polyoxymethylene (POM, polyacetal, | 24969-26-4 | 37 | 76.8 |
| polymethylene oxide) | | | |
| Polyvinylidene chloride (PVDC, Saran) | 9002-85-1 | 40.2 | 80 |
| Polyphenylene sulfide (PPS) | 26125-40-6 | 38 | 80.3 |
| Acrylonitrile butadiene styrene (ABS) | 9003-56-9 | 38.5 | 80.9 |
| Nylon 11 | 25587-80-8 | 35.6 | 82 |
| Polycarbonate (PC) | 24936-68-3 | 44 | 82 |
| Polyvinyl fluoride (PVF) | 24981-14-4 | 32.7 | 84.5 |
| Polyvinyl chloride (PVC) | 9002-86-2 | 37.9 | 85.6 |
| Nylon 8,8 | — | 34 | 86 |
| Nylon 9,9 | — | 34 | 86 |
| Polystyrene (PS) | 9003-53-6 | 34 | 87.4 |
| Polyvinylidene fluoride (PVDF) | 24937-79-9 | 31.6 | 89 |
| Poly n-butyl methacrylate (PnBMA) | 25608-33-7 | 29.8 | 91 |
| Polytetrafluoroethylene | 24980-67-4 | 26.5 | 92 |
| Nylon 10,10 | — | 32 | 94 |
| Polybutadiene | 9003-17-2 | 29.3 | 96 |
| Polyehylene (PE) | 9002-88-4 | 31.6 | 96 |
| Polychlorotrifluoroethylene (PCTFE) | 9002-83-9 | 30.8 | 99.3 |
| Polypropylene (PP) | (a) | 30.5 | 102.1 |
| Polydimethylsiloxane (PDMS) | 9016-00-6 | 20.1 | 107.2 |
| Poly t-butyl methacrylate (PtBMA) | 25189-00-9 | 18.1 | 108.1 |
| Fluorinated ethylene propylene (FEP) | 25067-11-2 | 19.1 | 108.5 |
| Hexatriacontane | 630-06-8 | 20.6 | 108.5 |
| Paraffin | 8002-74-2 | 24.8 | 108.9 |
| Polytetrafluoroethylene (PTFE) | 9002-84-0 | 19.4 | 109.2 |
| Poly(hexafluoropropylene) | — | 16.9 | 112 |
| Polyisobutylene (PIB, butyl rubber) | 9003-27-4 | 27 | 112.1 |

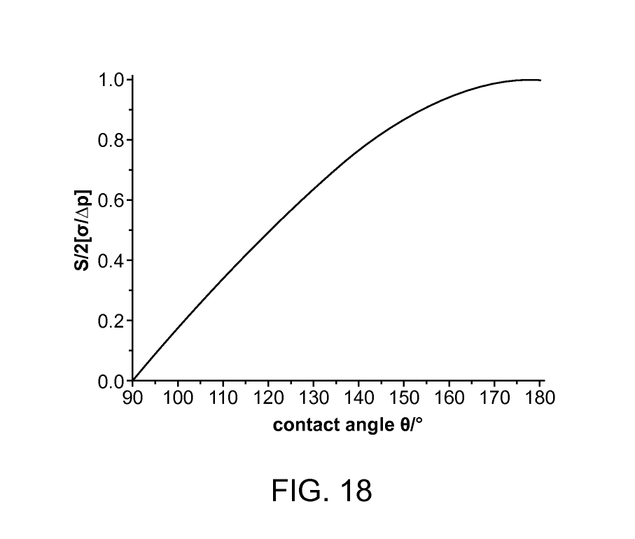

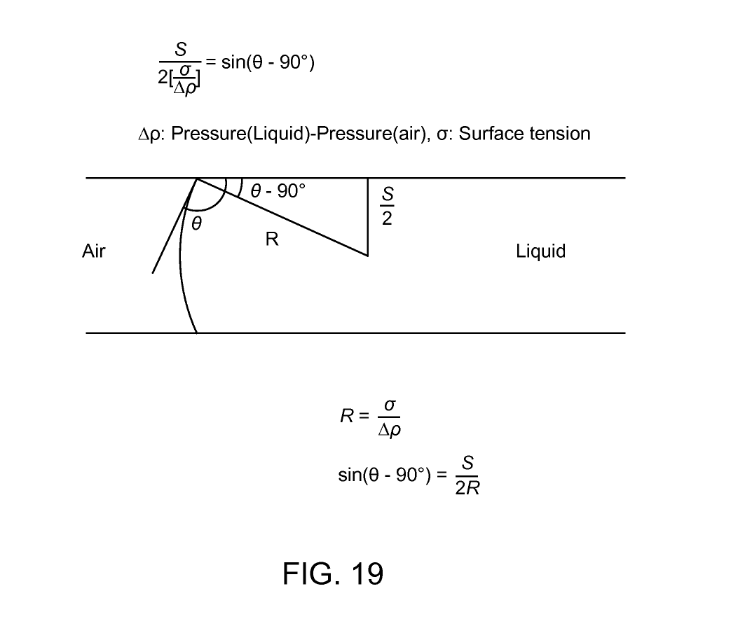

The hydrophobicity of the opposing hydrophobic or superhydrophobic surfaces and the maximum distance these surfaces can be spaced (and any minimum distance those surfaces should be spaced) to inhibit protrusion of water into the space can be quantified (i.e., the hydrophobicity/separation distance relationship can be characterized) using the formulas shown in FIGS. 18 and 19. These formulas also describe the expelling of liquid surface from high curvature location (e.g., a corner) in terms of contact angle.

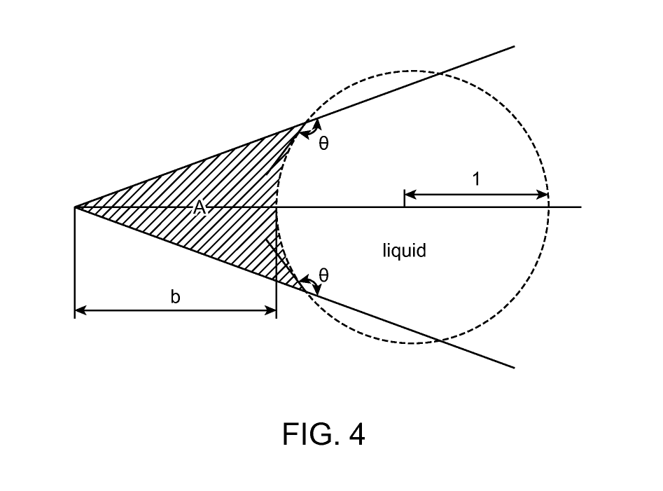

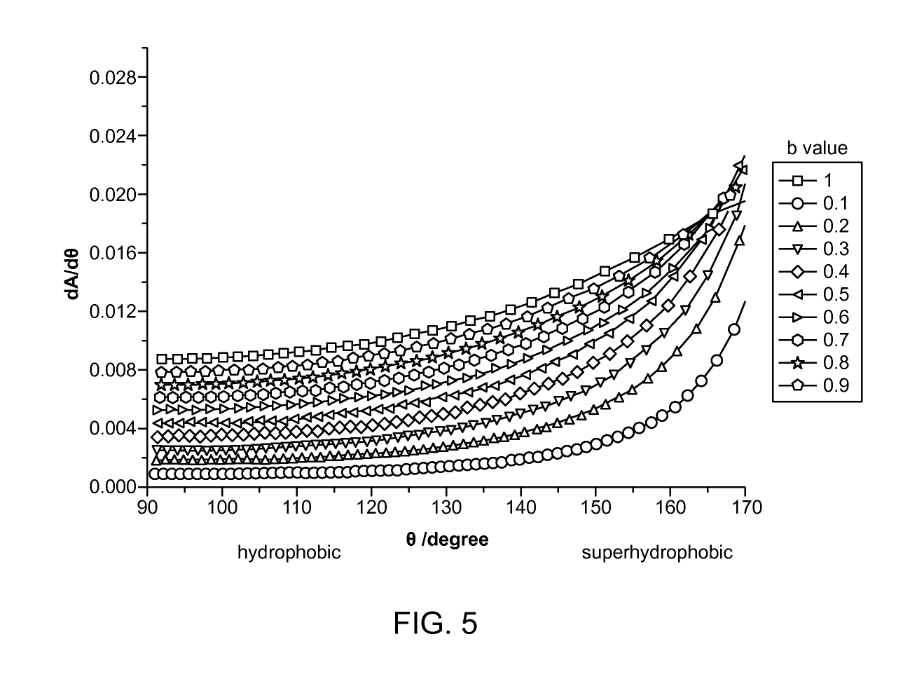

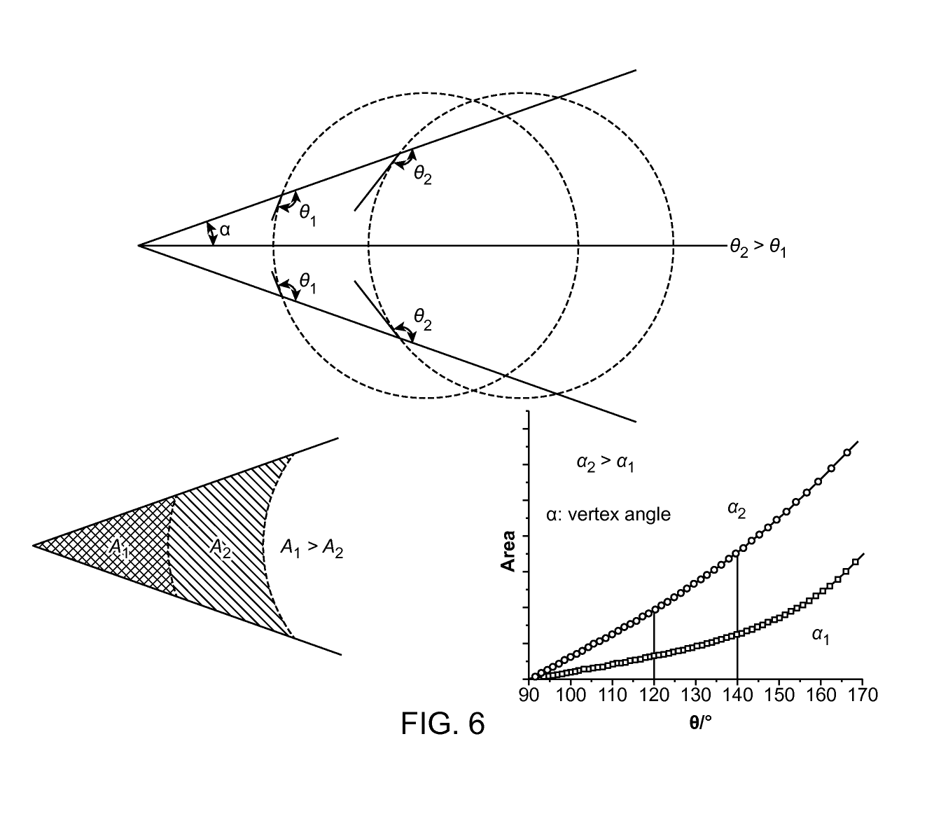

Referring to FIG. 4, FIG. 5 and FIG. 6, as contact angle θ increases, void for air passage increases as the liquid air interface is pushed further away from the corner of the vertex (high curvature location). At θ greater or equal to 140° change becomes more pronounced. In FIG. 4:
A is area in red, its size depends on contact angle θ
The length unit is the radius of circle
b: The distance from vertex of corner to the liquid surface

dA/dθ=(0.004b+0.0005)exp[(0.017b²−0.0392b+0.0317)θ]

The rate of void size increases exponentially with contact angle.

A catheter described herein can be used for any application in which improving liquid flow through a catheter or similar structure is desired (e.g., by reducing frictional resistance between the liquid and the interior of the catheter or other structure). In addition to urinary catheterization, examples of such applications include dentistry when, for example, draining the water jet tube after use, and liquid transfer in confined space under low gravity or zero gravity conditions. In the catheters described herein, the hydrophobic material and the modification impart antimicrobial properties to the interior surface. The hydrophobic (e.g., superhydrophobic) coating may be used to coat the entire interior surface of a catheter including the modification, providing an anti-microbial effect as well as improving fluid through. In the present invention, stagnation of fluid or impediment of fluid flow through a catheter is decreased or eliminated, which also provides an anti-microbial effect.

Methods of using the catheters described herein are encompassed by the present invention. For example, a method of flowing liquid through a catheter can include providing a catheter as described herein, the catheter having opposing ends, an inlet orifice at one end and an outlet orifice at the other end; and causing liquid flow through the inlet orifice. In this method, air is trapped in the air flow channel and liquid flows through the liquid flow channel and not through the air flow channel. In another example, a method of flowing liquid through a catheter can involve catheterizing a patient. In this method, catheterizing the patient includes positioning the inlet orifice in the patient's urethra, the outlet orifice in fluid communication with a urine collection device.

### EXAMPLES

The present invention is further illustrated by the following specific examples. The examples are provided for illustration only and should not be construed as limiting the scope of the invention.

### Example 1

### Better Catheter Design with Improved Liquid Flow Through Tubes

It is impossible for some post-surgical patients and stroke victims to pass urine on their own. When this happens, a catheter is used to pass the urine for the patient. The catheter is connected to the patient's bladder on one opening and to a drainage bag on the other. The drainage bag collects the discharged urine, which flows from the bladder through the catheter and into the drainage bag slowly and intermittently. When the urine flows through the tube, it is forced to travel around air bubbles that are confined within the tube. As the urine squeezes through the gap between the air and the wall of the tube, the air bubbles slowly rise and allow urine to travel downwards. In this classic example of film flow, resistance is significant; due to this resistance the urine may become stagnant or even flow back into the tube, resulting in painful pressure on a sensitive part of the body.

Bacterial colonization of catheters is common due to backflow and stagnation. Each day of catheter use increases the chance for the appearance of bacteria in the urine by five percent. These infections can have serious consequences, including death. Infections can be prevented by maintaining a closed drainage system, keeping high infection control standards, and preventing backflow from the catheter bag. Preventing the backflow from the catheter bag in a closed drainage system requires improved liquid flow through closed tubes in the presence of confined bubbles.

Experiments were run on a variety of rigid tubes with a super-hydrophobic nanoparticle coating covering their interior surface. Each tube features a different cross-sectional geometry and the tubes were tested to determine the most effective drainage system. Due to the super-hydrophobic coating, the liquid will tend to stay away from the tube's surface and liquid will not occupy corners. As a result, liquid will tend to move in the central region of the tube while the air counter-flow will use the passage near the corners. The outcome was impressive, with bubbles no longer obstructing the liquid flow. The combination of the modified cross sectional geometry in conjunction with the hydrophobic coating effectively prevented the confined bubbles from obstructing liquid flow in the tubes. The applications to the improvement of urinary catheter design are also discussed.

The present study focuses on preventing backflow from the catheter bag in a closed system. It is difficult for liquid to flow efficiently in a drainage tube system since both ends of the tube are closed, with one end attached to the bladder and the other connected to drainage bag. The end connected to the drainage bag will only open when there is sufficient hydrostatic pressure built up to open it; this is done intentionally in order to maintain a closed system to help inhibit infections. The physics of the flow through in the tubes in these experiments were approximated by closing both ends.

Few studies have been made for the situation where both ends of the tube are closed. The typical urinary catheter has a balloon attachment at the end that is inserted into the bladder. After the catheter is inserted, the balloon is filled with sterile water, which prevents the catheter from slipping out of the bladder. At this moment, there is only water and air in the catheter tube. Once the catheter enters the bladder, urine will begin to drain down the tube via gravity. It flows through the catheter and into the sterile drainage bag. This is a closed system with the bladder on one side and the drainage bag on the other. Only gravity is at work here, pulling the urine out of the bladder, town the catheter tube, and into the drainage bag. Urine discharge is small and intermittent, and the drainage tube is mostly occupied by an air bubble which confines the liquid flow. Backflow occurs when liquid flows through a closed tube in the presence of confined bubbles. Backflow is known as “reflux”—a dangerous condition in which overfilling of the bladder can lead to backflow into the kidneys.

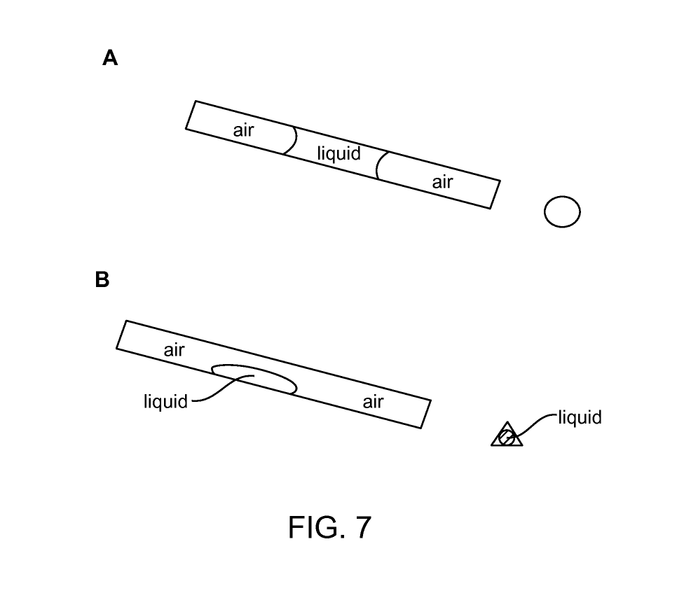

As the urine squeezes through the gap between the air bubble and the wall of the tube, the bubble slowly rises and allows the urine to pass (see FIG. 7A). The resistance of this film flow is significant, which causes the urine flow to stagnate or in some cases flow backward, resulting in painful pressure on a sensitive part of the body. Preventing backflow and flow stagnation using a control valve or pump is not feasible; increasing the tube's pressure would result in pressurized pushing against a sensitive organ. The modified catheters described herein allow the liquid to flow without being impeded by gas bubbles confined in the tube. One approach is to use a tube with a hydrophobic coating and an angular cross section. The hydrophobic coating causes the liquid to tend to stay away from the tube's surface, while the angular geometry prevents liquid from occupying the corners of the tube. As a result, the liquid tends to move into the central region of the tube while the air moves in the opposite direction along the corners. (FIG. 7B). FIG. 7B shows flow in a closed tube with a hydrophobic coating and an angular cross section. The hydrophobic coating causes the liquid to tend to stay away from the tube's surface, while the angular geometry prevents liquid from occupying the corners of the tube. As a result, the liquid tends to move into the central region of the tube while the air moves in the opposite direction along the corners.

The natural flow rates were studied by watching how the liquid moves through the tube. In this study, only one bolus of liquid was chosen for measurement. To maintain commercial applicability in the experiment, only tubes whose cross sections most closely resemble that of tubes which are already used commercially were used. The objective is to allow the liquid to flow without being impeded by air bubbles confined in the tube. The use of tubes that feature differing cross sections to determine how to best avoid this impedance is described herein.

### Materials and Methods

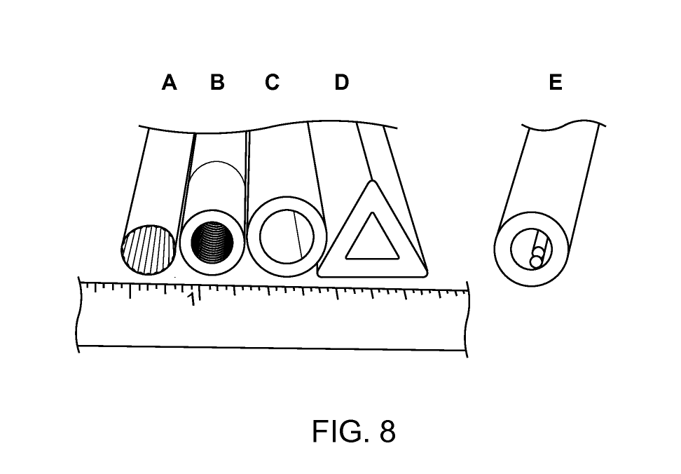

The experiment was conducted with rigid tubes featuring five different cross sections: a fin walled cross section, a circular tube with a rod placed inside, a circular cross section, and a triangular cross section. (FIGS. 8A-E). Each of the five various cross sectional areas were first tested with the super-hydrophobic coating, and then tested without the super-hydrophobic coating. The group without the super-hydrophobic coating served as the control group.

The first approach is to study the tubes that feature hydrophobic coating and sharp angular cross sections. The hydrophobic coating causes the liquid to stay away from the tube's surface, while the angular geometry prevents liquid from occupying the corners of the tube. As a result, the liquid tends to move into the central region of the tube while the air moves in the opposite direction along the corners. With the hydrophobic coating, the liquid tends to stay away from the tube's surface and, together with geometry modifications with embedded small curvatures, liquid will not occupy those corners. As the result, liquid will tend to move in the central region of the tube while the air counter-flow uses the passage near those corners.

In the study, approximately 0.1 ml of water is added to a rigid tube via suction (the tube is inserted in a pool of water and suction is applied to draw water into the tube). As aforementioned, the tubes featured different geometries; the triangular tube featuring an inner area of 0.1 cm² and the circular tubes featuring an inner cross-sectional area of 0.08 and 0.18 cm² respectively. The flow rate was measured via video tracking the front portion of a bolus of liquid (which was approximately 1 cm in length) as it traveled through a 31 cm section of tubing.

The tubes were coated with a proprietary superhydrophobic coating from the Industrial Technology Research Institute (ITRI) in Taiwan. The coating is a sol-gel coating containing trimethylsiloxane-silica nanoparticles. It has no known toxicity for external use, and its basic technology is similar to the coating composition of US Patent US2008/0221009A1.

Water was placed in each tube and then both ends of the tube were closed in order to replicate the affect of the tube being connected to a drainage bag and the human body. The tube was oriented at three different angles, 30°, 45°, and 90°, respective to a horizontal plane, to see if angle of inclination had any effect on the travel time. A timer was used to determine the time it took for the water to flow one foot; the data was then recorded. Each trial was repeated several times and the average was calculated to determine the flow time of each tube under different conditions. The process was repeated under a non-closed condition in which the tubes were open at the ends in order to test the flow of the liquid under normal air pressure. The results are discussed in the following section.

### Results and Discussion

There is a dramatic difference between the travel time for the tubes without the super-hydrophobic coating, which ranges from 0.24-384.32 seconds, and the tubes with the super hydrophobic coating, ranging from 0.24-3.20 seconds. No flow was observed with or without a super-hydrophobic coating for a tube with a circular cross section. The tubes that were not coated with the super hydrophobic coating and that were closed at both ends showed no flow movement regardless of the tube shape or the angle positioned. As expected, the angle of inclination of the tube affected the flow rate. Overall, the tubes that were angled 90° relative to a horizontal plane outperformed those inclined at 45° and 30°.

TABLE 2
The time taken for water to flow one foot through hydrophobic coated tubes of varying cross sections
with both ends open and both ends closed was measured. NF means that no flow was observed.

| | Closed both ends | | | Open both ends | | |
|---|---|---|---|---|---|---|
| | Angle of inclination (degrees) | | | Angle of inclination (degrees) | | |
| Cross section | 30 | 45 | 90 | 30 | 45 | 90 |
| (coated) | Average Time to flow 1′ (seconds) | | | | | |
| Circular with | 3.2 ± 0.35 | 1.87 ± 0.55 | 1.18 ± 0.15 | 0.95 ± 0.12 | 0.72 ± 0.04 | 0.5 ± 0.07 |
| Rod | | | | | | |
| Triangular | 2.37 ± 0.29 | 1.51 ± 0.24 | 1.47 ± 0.15 | 1.52 ± 0.27 | 1.31 ± 0.25 | 1.33 ± 0.24 |
| Circular | NF | NF | NF | 1.28 ± 0.20 | 0.62 ± 0.09 | 0.52 ± 0.07 |
| Fin-Walled | 2.48 ± 0.49 | 0.93 ± 0.07 | 1.08 ± 0.27 | 0.37 ± 0.06 | 0.45 ± 0.18 | 0.24 ± 0.04 |

TABLE 3
The time taken for water to flow one foot through uncoated tubes of varying cross
sections with both ends open and both ends closed was measured. NF means that no flow
was observed.

| | Closed both ends | | | | | |
|---|---|---|---|---|---|---|
| | Angle of inclination | | | Open both ends | | |
| | (degrees) | | | Angle of inclination (degrees) | | |
| Cross section | 30 | 45 | 90 | 30 | 45 | 90 |
| (uncoated) | Average Time to flow 1′ (seconds) | | | | | |
| Circular with | NF | NF | NF | 21.05 ± 8.8 | 18.28 ± 3.5 | 14.01 ± 6.6 |
| Rod | | | | | | |
| Triangular | NF | NF | NF | 384.32 ± 41.0 | 18.98 ± 3.1 | 3.86 ± 0.3 |
| Circular | NF | NF | NF | 82.12 ± 10.9 | 6.7 ± 0.99 | 1.49 ± 0.15 |
| Fin-Walled | NF | NF | NF | 1.15 ± 0.04 | 0.34 ± 0.05 | 0.29 ± 0.11 |

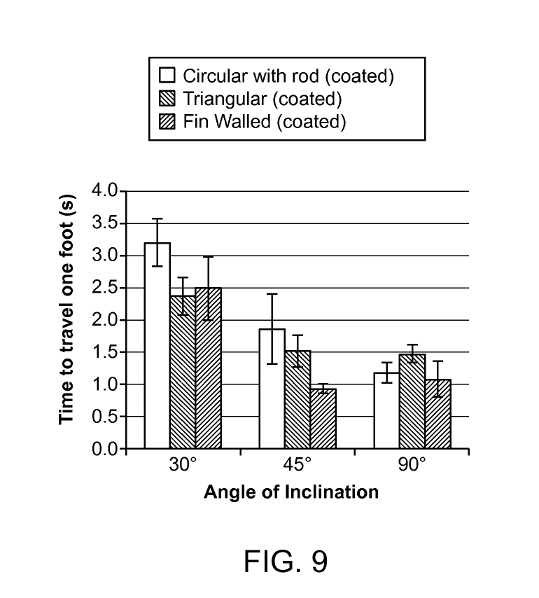

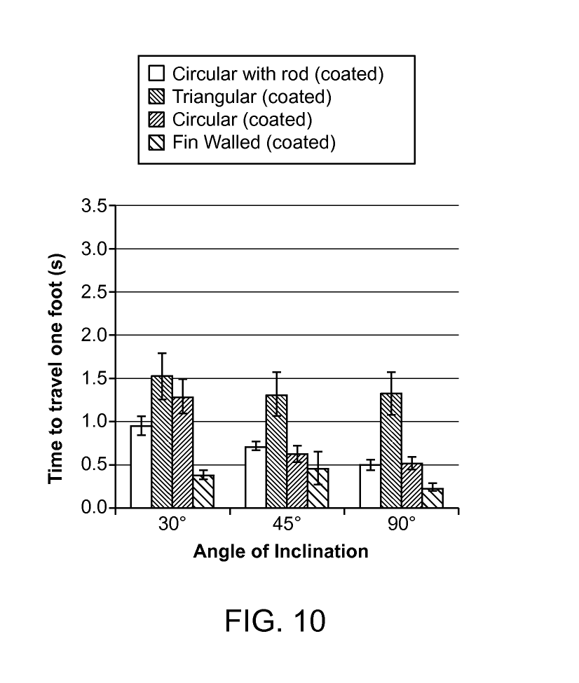

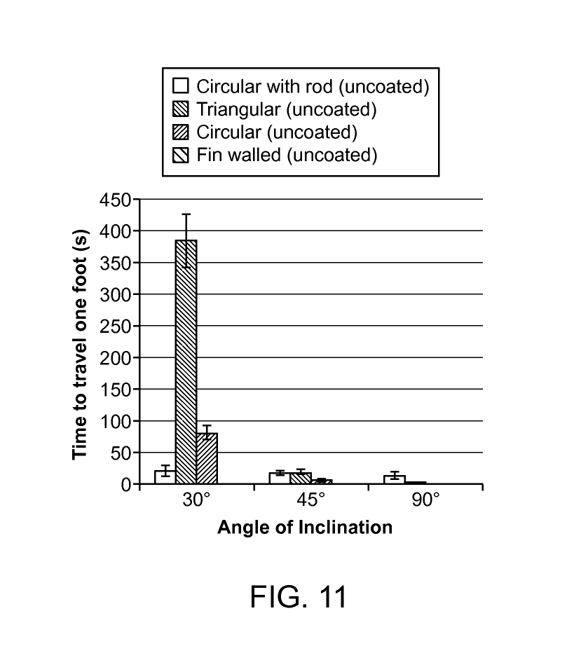

Referring to FIG. 9, the time taken for the fluid to travel one foot in a tube with both ends closed was measured in coated tubes of various cross section at various angles of inclination respective to the horizontal plane. No flow was observed in the tube with a circular cross-section. Referring to FIG. 10, the time taken for the fluid to travel one foot in a tube with both ends open was measured in coated tubes of various cross section. Notice that the time taken drops significantly for tubes of the same geometry. Referring to FIG. 11, The time taken for the fluid to travel in one foot in a tube with both ends open was measured for uncoated tubes of various cross section. In general, the time taken for the fluid to travel increased by a significant amount without hydrophobic coating. With the exception of the tube with a circular cross-sectional area, the coated tubes all performed roughly equivalently in the both-ends-closed case. The triangular coated tube was slightly worse for the both-ends-open case.

In summary, the use of a superhydrophobic tube in conjunction with various cross sectional geometries (fin-walled, triangular, and circular) is presented to determine improved catheter and drainage bags for discharging urine or other bodily fluids, and to improve liquid drainage efficacy in the presence of confined bubbles in a closed tube. This advance has the potential to greatly reduce the rate of urinary tract infections during catheterization, currently the most common healthcare-associated infection worldwide. Additional applications of this technology exist in medicine and liquid transport in space applications.

### Example 2

### Reducing Urinary Tract Infections with Superhydrophobic-Textile-Lined Catheters

### SUMMARY

An innovative indwelling urethral catheter that minimizes urine backflow was developed. Adding a superhydrophobic element to alter the inner surface properties of a catheter resulted in an improved catheter design that prevents confined air bubbles from obstructing liquid flow.

### Methods

Obstruction of the liquid flow could be remedied by that fact that an air passage is inherently created inside the tube when a superhydrophobic coated fabric is inserted into the tube. We test two different superhydrophobic textiles inside of a catheter tube in an effort to determine how superhydrophobic fabrics affect liquid flow through closed tubes. These textiles, known respectively as Polypropylene spunband fabric and Polypropylene meltblown fabric, will be inserted into the catheter tube for experimentation. It's expected that these thin (15-25 microns) superhydrophobic textiles, developed by Zimmermann (Mueller et al. Journal of Urology 2005; 173:490-2) and Dr. Gajanan Bhat of the Nonwoven Materials Research Laboratory (UTNRL) at the University of Tennessee, Knoxville, will allow sufficient air passage through the space within the air-breathable fabric such that little or no additional geometric modification shall be required and liquid flow will improve significantly.

### Results

Through experimentation we found that the superhydrophobic-coated-textile inserts enabled liquid flow through a closed-system catheter tube. The standard closed tube system showed no liquid flow, whereas the tube containing the superhydrophobic fabrics demonstrated a much improved flow rate in the closed tube system. The air bubbles that currently prevent liquid flow in catheter tubes were displaced by the liquid very efficiently due to the addition of superhydrophobic-coated fabric.

### Example 3

### Superhydrophobic-Textile-Lined Catheters

The specific goal of this research work was to develop and test the feasibility of adding a superhydrophobic element to alter the inner surface properties and test the hypothesis that this can result in an improved catheter design that prevents confined air bubbles from obstructing liquid flow.

### Method

In order to create flow in a closed tube, several types of super-hydrophobic-coated textiles are inserted into a series of tubes.

TABLE 4
Fabric samples were produced at the University of Tennessee
Nonwovens Research Laboratory (UTNRL), Knoxville, TN (Mueller
et al. Journal of Urology 173: 490-492, 2005). The target
was to produce fabrics with similar weight per unit area,
but different permeability characteristics.

| | Basis | | |
|---|---|---|---|
| | Weight | Thickness | Air Permeability |
| Samples | (g/m²) | (mm) | (ft³/min) |
| (A) Spunbond | 60 | 0.42 | 195 |
| PP | | | |
| (D)Meltblown | 30 | 0.33 | 99 |
| PP, 50 cm | | | |
| (B)Meltblown | 29 | 0.25 | 61 |
| PP, 15 cm | | | |
| (E)Meltblown | 32 | 0.48 | 104 |
| PLA, 15 cm | | | |

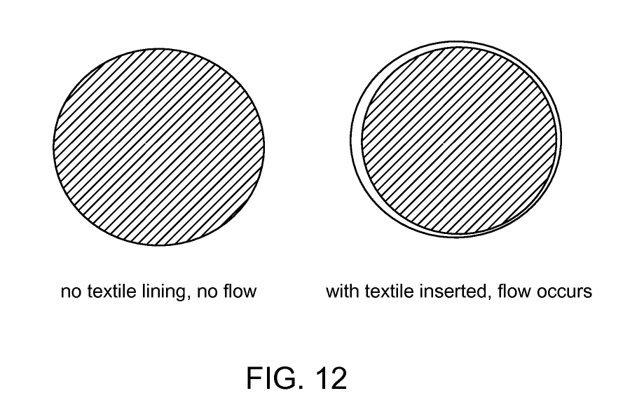

A variety of circular tubes were chosen for this experiment; some are rigid and some are flexible, all with differing lengths and inner diameters. Referring to FIG. 12, in the non-lined tubes, no flow is possible at 90 degrees. When the super-hydrophobic textile is inserted into the tubes, the water is able to flow. Each of the tubes were tested with and without the super-hydrophobic textile inserts. The group without the super-hydrophobic textile served as the control group. The tubes were oriented at six different angles, 15°, 30°, 45°, 60°, 75°, and 90° (vertical), to see if the angle of inclination had any effect on the flow rate. A 2.54 cm column of water was used for each experiment, and the time for the top of the water column to reach the bottom of the tube from the top of the tube is measured. A timer was used to determine the time elapsed for the 2.54 cm length of water to flow the entire length of the tubes. The tubes feature varying lengths to determine if length has any effect on flow rate. The data was recorded; each trial was repeated three times, and the averages were calculated to determine the flow rate for each tube under differing conditions.

### Results

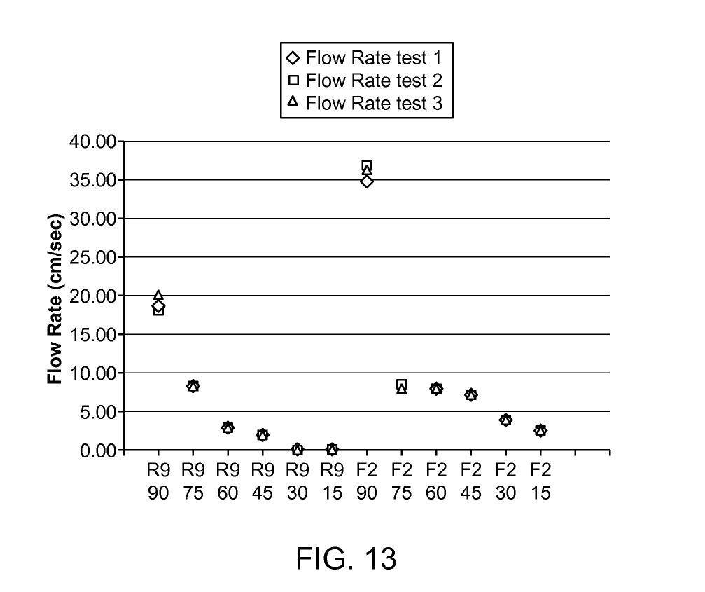

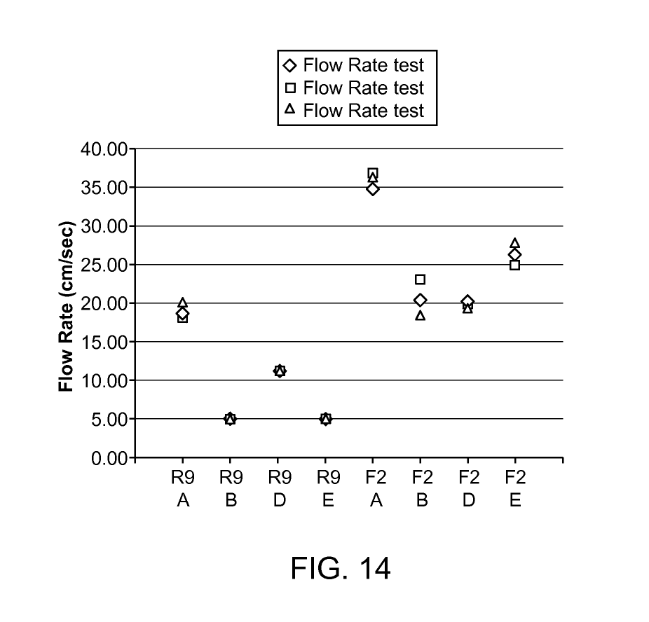

Herein (FIGS. 13 and 14) are presented the results for two of the tubes, both 0.635 centimeters in diameter, one rigid (107.32 cm in length) referred to from here on as “R9” and the other flexible (30.48 in length) referred to from here on as “F2”. Referring to FIG. 13, the effect of textiles on flow rate (speed) for rigid and flexible tubes is shown. Referring to FIG. 14, the effect of angle on flow rate (speed) is shown. This graph presents only the results for textile “A” for tubes “R9” and “F2” at all angles of inclination.

The results show that optimal flow occurs at a 90 degree angle of inclination regardless of the rigidity of the tube, that flexible tubes offer better flow rates than rigid tubes, and that Textile A offers the best flow rate improvement. Tube length does not appear to have any effect on the flow rate.

Through experimentation it was found that the superhydrophobic-coated-textile inserts enabled liquid flow through a closed-system catheter tube. The standard closed tube system showed no liquid flow, whereas the tubes containing the superhydrophobic fabrics demonstrated a much improved flow rate in the closed tube system. The air bubbles that currently prevent liquid flow in catheter tubes were displaced by the liquid very efficiently due to the addition of superhydrophobic-coated fabric. The Polypropylene superhydrophobic fabrics are non-toxic, easy to produce, and inexpensive to implement into current catheter designs.

### Example 4

### Reduction of Urinary Tract Infections Caused By Urethral Catheter through the Implementation of Hydrophobic Textile Coating and Other Geometrical Modifications

A catheter is a drainage-system used to drain the bladder to help prevent kidney damage and urinary tract infections when the bladder doesn't expel urine properly, otherwise understood as a case of urinary incontinence or urinary retention. The catheter is a plastic tube with one end attached to a drainage bag and the other end penetrating the urethra, through the valve and into the bladder. While in place, the bladder is no longer pressurized and the muscles no longer contract; instead, urinating is involuntary and sporadically drains through the catheter tube. Intermittent catheters are inserted and removed several times a day whereas indwelling catheters are inserted for days at a time. The indwelling catheter includes an additional part (being the balloon) to hold the tubing in place so that it does not slip out of the urethra. 80% of urinary tract infections associated with healthcare are caused by indwelling catheters, being more accountable for CAUTI than the intermittent catheters. Both catheter types are susceptible to causing UTIs. This study will focus on the CAUTI by experimentation with intermittent catheters.

CAUTIs are generally asymptomatic infections and reveal presence of bacteriuria or candiduria through urine cultures. Asymptiomatic CAUTIs are defined as the absence of frequency, dysuria, urgency and suprapubic tenderness. It is suggested that patients are not treated for CAUTIs by antimicrobial agents (antibiotics, antivirals, antifungals, and antiparasitics) while the catheter is in place because of the probable evolution of resistant flora. The greatest problem with CAUTI is the risks of pyelonephritis, cystitis, urethritis, bacteremia, and sepsis. One cause of UTIs in catheters is that the material is made of plastic tubing and as such, flora is able to adhere to the surface. Resistant flora colonization in CAUTI occurs due to the presence of bacterial biofilms forming adherence to the catheter tube. The protein of bacteriuria and candiduria are difficult to eradicate as long as the catheter remains in place. The natural mechanical flushing of urine and the shedding of epithelial cells lining the urinary tract prevent flora migration due to their sterilizing effects. Inserting a catheter obstructs these natural protective functions and permits the risk for infection. Accordingly, CAUTIs are a major reservoir of antibiotic-resistant organisms. Another cause of CAUTI is the obstruction of urine caused by confined air bubbles, which slow down and stagnate the urinary flow to the drainage bag from the bladder. With increase in pressure within the tube due to obstructed liquid, gravity is no longer pulled downwards into the drainage bag. When there is stagnant urine within the tube, new urine is unable to pass through the tube and so it flows backward, causing the bacteria-ridden (or fungi-ridden) urine to return into the urinary tract. Hydrophobic coating within the tube, being either textile-lining or a liquid-spray, can alter the surface properties to improve the current catheter design. The nature of hydrophobic material is to repel water, which would keep liquid away from the inner surface of the tube when inserted into the catheter tube. This would facilitate airflow by allowing sufficient air passage between the urine and the tube, so the urine may flow in one direction and not become obstructed by confined by air bubbles. The type of hydrophobic textile-lining used in this study is polypropylene fabrics, which are non-toxic, easy to produce, and inexpensive to implement into current catheter designs.

In order to promote improved urination flow through the catheter tube for the reduction of UTIs, the catheter tube must not be obstructed by fluid. At any amount that passes through the closed-system catheter tube, there should be weight applied to the static fluid column in order to distribute the change in pressure throughout the tube. This would entail the principle of transmission of fluid-pressure; Pascal's Law expresses this principle by:

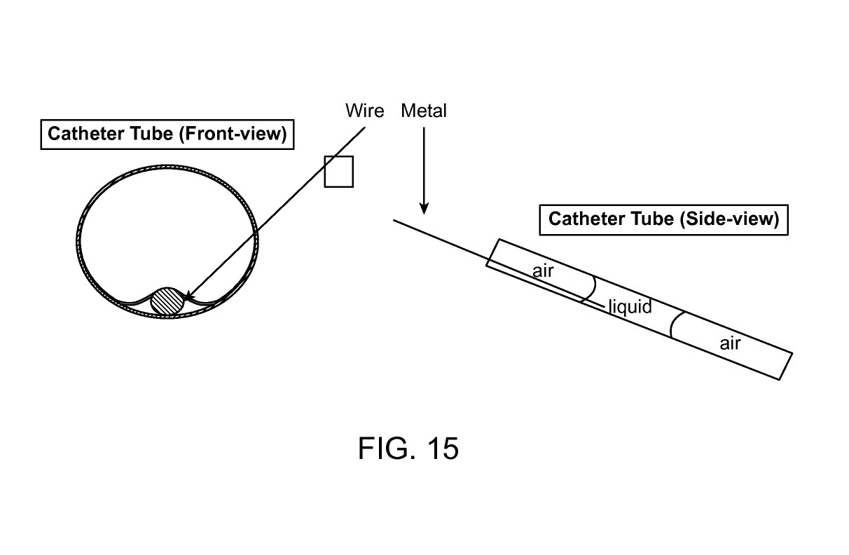

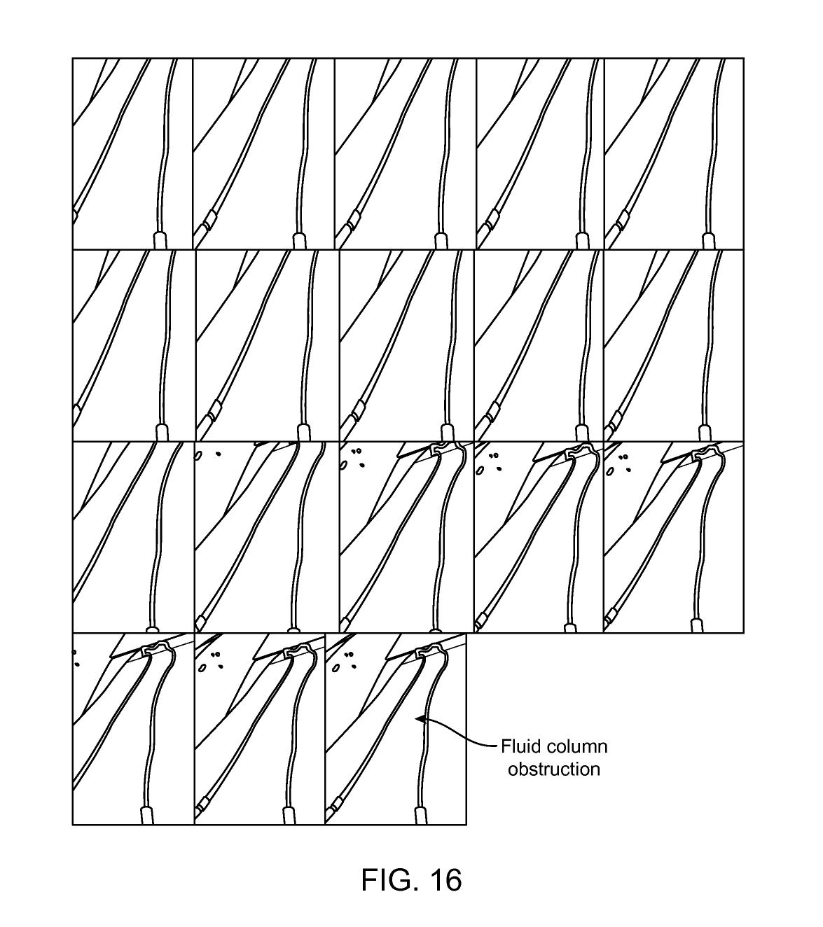

ΔP=ρg Δh

Where: ΔP is the hydrostatic pressure (difference in pressure of fluid column at 2 points due to the fluid's weight); p is the fluid density; g is gravitational acceleration (Earth's being 9.81 m/s2); and Δh is the height of the fluid column. In order to apply this principle, qualitative measurements in a basic study were performed. Two intermittent catheter systems were used in experiment. One of the catheter systems included an inserted wire metal. Referring to FIG. 15, the metal wire is placed into the catheter tube to pierce the fluid column and release the surface tension for dispersal of pressure. Referring to FIG. 16, the catheter with metal wire is shown on the right and the catheter without the metal wire is shown on the left. It can be observed that the wire metal allows for dispersal of the fluid column, whereas the lack of wire metal in the catheter tube has caused the fluid to become obstructed in the form of a column. Each catheter was placed at the same angle, height, and length. Liquid was leaked into tube as closed-system at same volumes. Liquid stagnates in the tube with a closed end, but begins to move once disturbed by a metal wire. This displays the change in surface tension of the destabilized liquid/air interface.

### Other Embodiments

Alterations and improvements within the scope of the invention may be made to part or all of the embodiments of the invention as herein described. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

1. A catheter comprising an elongate body defining a continuous enclosed liquid flow channel, at least a portion of the liquid flow channel comprising a modification having a first surface and a second surface and an included volume between the first surface and the second surface, at least a portion of the included volume defining an elongated air flow channel, at least a portion of the first surface and the second surface coated with a hydrophobic material, whereby the air flow channel excludes liquid flow but is in fluid communication with the liquid flow channel.

2. The catheter of claim 1, wherein at least one of the modification and the hydrophobic material have anti-microbial properties.

3. The catheter of claim 1, wherein the hydrophobic material is superhydrophobic.

4. The catheter of claim 1, wherein the continuous enclosed liquid flow channel extends for the length of the elongate body.

5. The catheter of claim 1, wherein the air flow channel is contiguous with the continuous liquid flow channel.

6. The catheter of claim 1, wherein the elongate body defines an inner wall and an elongated rod is disposed within the continuous liquid flow channel and is attached to the inner wall, the rod being coated at least in part with the hydrophobic material.

7. The catheter of claim 1, wherein the modification is a porous material, portions of the porous material defining the first and second surfaces and the air flow channel.

8. The catheter of claim 7, wherein the porous material is a textile of interwoven fibers, the interwoven fibers defining the first and second surfaces and the air flow channel.

9. The catheter of claim 7, wherein the porous material is a carbon foam.

10. The catheter of claim 7, wherein the porous material defines air passages having a size permitting the passage of air molecules and preventing the passage of water molecules.

11. The catheter of claim 1, wherein the elongate body defines an inner wall, the inner wall having angular portions, the angular portions including the first and second surfaces.

12. The catheter of claim 11, wherein the elongate body is triangular in cross-section, the corners of the triangle defining the angular portions.

13. The catheter of claim 11, wherein the elongate body is circular in cross-section.

14. The catheter of claim 1, wherein the elongate body includes at least a first fin extending radially inward from the inner wall, portions of the at least first fin including at least one of the first and second surfaces.

15. The catheter of claim 1, wherein the inner wall comprises at least one elongated groove including the first and second surfaces, the groove and the first and second surfaces defining the air flow channel.

16. The catheter of claim 14, wherein at least a portion of the inner wall adjacent the at least one fin includes the hydrophobic coating, whereby the included volume between the at least one fin and the inner wall defines the air flow channel.

17. The catheter of claim 14, further comprising at least a second fin adjacent and parallel to the at least first fin, the first fin defining one of the first and second surfaces, the at least second fin defining at least one of the first and second surfaces, the first fin and the second fin defining an enclosed space between the at least first and second fins, the enclosed space defining in part the air flow channel.

18. The catheter of claim 1, wherein the catheter is a drainage tube.

19. The catheter of claim 1, wherein the catheter is a urinary catheter, and further comprises an apparatus for retention of the catheter within a subject's urethra.

20. The catheter of claim 19, wherein the catheter is dimensioned for insertion into a patient's urethra, having a diameter of about 0.15 to about 0.51 inches.

21. The catheter of claim 19, wherein the catheter defines proximal and distal ends, the retention apparatus being at the proximal end and a urine collection assembly in fluid communication with the distal end.

22. The catheter of claim 1, wherein the catheter is a Foley catheter.

23. A urinary catheter comprising an elongate body defining a continuous enclosed liquid flow channel, at least a portion of the liquid flow channel comprising a modification having a first surface and a second surface and an included volume between the first surface and the second surface, at least a portion of the included volume defining an elongated air flow channel, at least a portion of the first surface and the second surface coated with a hydrophobic material, whereby the air flow channel excludes liquid flow but is in fluid communication with the liquid flow channel.

24. The urinary catheter of claim 23, wherein the catheter further comprises an apparatus for retention of the catheter within a subject's urethra.

25. The urinary catheter of claim 24, wherein the catheter is dimensioned for insertion into a patient's urethra diameter of about 0.15 to about 0.51 inches.

26. The urinary catheter of claim 24, wherein the catheter defines proximal and distal ends, the retention apparatus being at the proximal end and a urine collection assembly in fluid communication with the distal end.

27. A method of flowing liquid through a catheter comprising: providing the catheter of claim 1, the catheter further comprising opposing ends, an inlet orifice at one end and an outlet orifice at the other end; and causing liquid flow through the inlet orifice, wherein air is trapped in the air flow channel and liquid flows through the liquid flow channel and not through the air flow channel.

28. The method of claim 27, wherein at least one of the modification and the hydrophobic material have anti-microbial properties.

29. The method of claim 27, further comprising catheterizing a patient, wherein catheterizing the patient comprises positioning the inlet orifice in the patient's urethra, the outlet orifice in fluid communication with a urine collection device.
